(19) United States
(12) Patent Application Publication
(10) Pub. No.: US 2023/0380788 A1
(43) Pub. Date: Nov. 30, 2023

(54) INFORMATION PROCESSING METHOD, MEDICAL IMAGE DIAGNOSTIC APPARATUS, AND INFORMATION PROCESSING SYSTEM FOR PROCESSING METAL ARTIFACT IMAGES

(71) Applicant: CANON MEDICAL SYSTEMS CORPORATION, Tochigi (JP)

(72) Inventors: Tzu-Cheng LEE, Vernon Hills, IL (US); Jian ZHOU, Vernon Hills, IL (US); Liang CAI, Vernon Hills, IL (US); Zhou YU, Vernon Hills, IL (US)

(73) Assignee: CANON MEDICAL SYSTEMS CORPORATION, Tochigi (JP)

(21) Appl. No.: 17/825,650

(22) Filed: May 26, 2022

(51) Int. Cl. A61B 6/00; A61B 6/03

(52) U.S. Cl. CPC A61B 6/5205; A61B 6/032; G06T 11/008

(57) ABSTRACT

An information processing method processes an x-ray image including the steps of: obtaining first lower-radiation dose three-dimensional image data during a first scan of a patient; and detecting, using a trained neural network, a presence of an artifact (e.g., a metal artifact) in the first lower-radiation dose three-dimensional image data. An information processing apparatus includes processing circuitry for performing the detection method, and computer instructions stored in a non-transitory computer readable storage medium cause a computer processor to performing the detection method.

### FIELD

Embodiments described herein relate generally to an information processing method, a medical image diagnostic apparatus, and an information processing system.

### BACKGROUND

A medical image acquired from a subject by a medical image diagnostic apparatus (e.g., an x-ray computed tomography (CT) apparatus) may include artifacts caused by metallic implants (e.g., pacemakers, wires, surgical clips, coil or dental fillings) which may appear as strong streaks on the images, which degrade the image quality and may decrease the diagnostic value of the examination. Metal artifacts are caused by a combination of multiple mechanisms including photon starvation (major), beam hardening (major), scattering, partial volume effects, under-sampling, and patient motion.

Known systems utilize projection-based metal artifact reduction (MAR) to improve image quality and recover information about underlying structures. The projection-based MAR algorithms act in projection space and replace corrupted projections caused by metal with information from neighboring uncorrupted projections. MAR algorithms primarily suppress artifacts that are due to photon starvation. Projection-based MAR algorithms often applied retrospectively after radiologists or technicians viewing reconstructed images and decide to apply the correction on it. Without clear information for predicting the metal artifact, most of CT MAR workflows requires manual inspection for metal artifacts. Manual image-quality checking workflows not only takes time but may reconstruct redundant images which also waste processing time and computational power. Furthermore, manual processes involve human error in finding metal artifacts that may decrease the overall diagnostic efficiency.

Computed tomography scout view (sometimes referred to as a prescan or a scanogram) is a mode of operating a CT system. It usually is applied before a major (or full/diagnostic) scan to provide at least one of: (1) assisting in patient centering and positioning, (2) allowing selection of anatomical targets for the following major (or full) CT scan, and (3) defining tube current modulation schemes. However, a traditional 2D scout scan usually shows only L-R and A-P direction of radiograph, is difficult to use to delineate soft tissue organs, and is sub-optimal for tube current modulation.

### BRIEF DESCRIPTION OF THE DRAWINGS

FIG. 1 is a block diagram of an exemplary configuration of an X-ray CT apparatus imaging a person as a subject according to an exemplary embodiment described below.

FIG. 2 is a block diagram of an exemplary configuration of information processing apparatus connected to an X-ray CT apparatus according to another exemplary embodiment described below.

FIG. 3A illustrates a general neural network to be trained according to an exemplary embodiment described below.

FIG. 3B illustrates a general convolutional neural network to be trained according to an exemplary embodiment described below.

FIG. 3C illustrates a general convolutional neural network having multiple connections between an input layer and a first hidden layer as part of a training process according to an exemplary embodiment described below.

FIGS. 4A-4C illustrate an x-ray emitter and an x-ray detector rotating about a patient to be imaged at multiple as part of a lower radiation dose three-dimensional scout scan (or preliminary scan) (e.g., using a 180 degree half reconstruction or a 360 degree full reconstruction) before performing a higher radiation dose main scan (or diagnostic scan).

FIG. 4D illustrates the combined process of FIGS. 4A-4C but with partial dashed lines indicating that the scout scan also can be performed as a sparse scan.

FIG. 5 illustrates a trained neural network receiving first lower-radiation dose image data from at least one angle during a first scan (e.g., a scout scan) of a patient.

FIG. 6A illustrates the training of a binary classifier neural network that is trained to detect the presence of an artifact in first lower-radiation dose image data obtained from at least one angle during a first scan.

FIG. 6B illustrates the training of a neural network (e.g., an autoencoder) that is trained to detect the presence of an artifact in first lower-radiation dose image data obtained from at least one angle during a first scan.

FIG. 7 illustrates part of an image processing method whereby the presence of a detected artifact causes automatic artifact reduction processing to be performed (and by-passed if no artifact is detected).

FIGS. 8A-8F shows a set of queues that can be used by an automatic image processing system to process the scout scans and main scans of FIG. 7.

### DETAILED DESCRIPTION

An information processing method of an embodiment is a method of processing an x-ray image, including, but not limited to: obtaining first lower-radiation dose three-dimensional image data during a first scan of a patient; and detecting, using a trained neural network, a presence of an artifact in the first lower-radiation dose three-dimensional image data. In at least one embodiment, the artifact is a metal artifact.

The disclosure herein also describes an information processing apparatus including processing circuitry and/or computer instructions stored in a non-transitory computer readable storage medium for performing the above-noted method.

Hereinafter, with reference to the accompanying drawings, an embodiment of an information processing method, a medical image diagnostic apparatus, and an information processing system will be described in detail.

In the present embodiment, X-ray CT will be described as an example of a medical image diagnostic modality. That is, in the present embodiment, an information processing method of information acquired by imaging performed by the X-ray CT will be described.

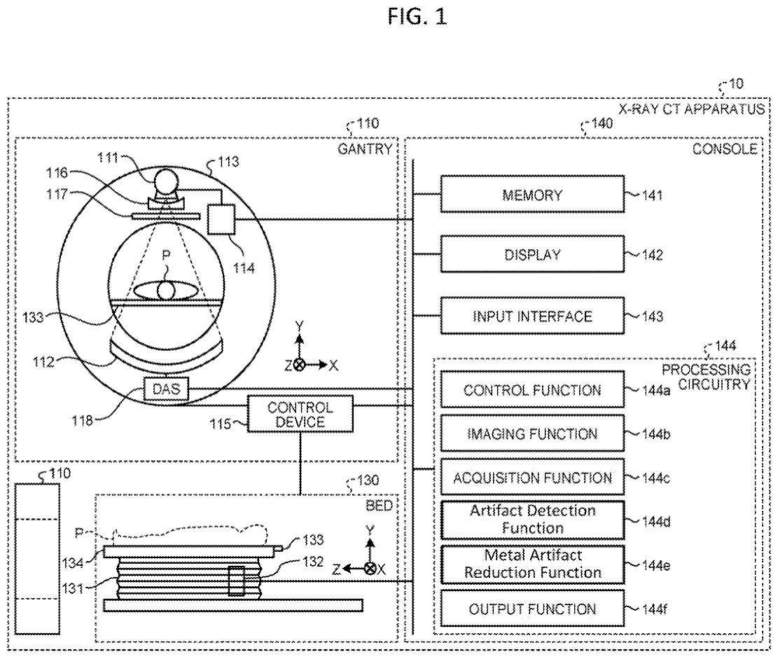

The X-ray CT is implemented, for example, in an X-ray CT apparatus 10 illustrated in FIG. 1. FIG. 1 is a block diagram illustrating an example of a configuration of the X-ray CT apparatus 10 according to a first embodiment. For example, the X-ray CT apparatus 10 has a gantry 110, a bed 130, and a console 140.

In FIG. 1, it is assumed that the longitudinal direction of a rotating shaft of a rotating frame 113 or a tabletop 133 of the bed 130 in a non-tilted state is a Z axis direction. Furthermore, it is assumed that an axial direction orthogonal to the Z axis direction and horizontal to a floor surface is an X axis direction. Furthermore, it is assumed that an axial direction orthogonal to the Z axis direction and perpendicular to the floor surface is a Y axis direction. Note that FIG. 1 illustrates the gantry 110 drawn from a plurality of directions for convenience of description and the X-ray CT apparatus 10 has one gantry 110.

The gantry 110 includes an X-ray tube 111, an X-ray detector 112, the rotating frame 113, an X-ray high voltage device 114, a control device 115, a wedge 116, a collimator 117, and a data acquisition system (DAS) 118.

The X-ray tube 111 is a vacuum tube having a cathode (filament) that generates thermoelectrons and an anode (target) that generates X-rays in response to a collision of thermoelectrons. The X-ray tube 111 emits the thermoelectrons toward the anode from the cathode by the application of a high voltage from the X-ray high voltage device 114, thereby generating the X-rays to be emitted to a subject P.

The X-ray detector 112 detects the X-rays emitted from the X-ray tube 111 and passed through the subject P, and outputs a signal corresponding to the dose of the detected X-rays to the DAS 118. The X-ray detector 112, for example, includes a plurality of detection element arrays in which a plurality of detection elements are arranged in a channel direction (channel direction) along one arc centered on a focal point of the X-ray tube 111. The X-ray detector 112, for example, has a structure in which the detection element arrays with the detection elements arranged in the channel direction are arranged in a row direction (slice direction and row direction).

For example, the X-ray detector 112 is an indirect conversion type detector having a grid, a scintillator array, and a photosensor array. The scintillator array has a plurality of scintillators. Each of the scintillators has a scintillator crystal that outputs light with a photon quantity corresponding to an incident X-ray dose. The grid has an X-ray shielding plate that is disposed on the surface of the scintillator array on an X-ray incident side and absorbs scatted X-rays. The grid may also be referred to as a collimator (a one-dimensional collimator or a two-dimensional collimator). The photosensor array has a function of converting light into an electrical signal corresponding to the amount of light from the scintillator, and has, for example, photosensors such as photodiodes. Note that the X-ray detector 112 may be a direct conversion type detector having a semiconductor element that converts the incident X-rays into electrical signals.

The rotating frame 113 is an annular frame that supports the X-ray tube 111 and the X-ray detector 112 so as to face each other and rotates the X-ray tube 111 and the X-ray detector 112 by the control device 115. For example, the rotating frame 113 is a casting made of aluminum. Note that the rotating frame 113 can further support the X-ray high voltage device 114, the wedge 116, the collimator 117, the DAS 118 and the like, in addition to the X-ray tube 111 and the X-ray detector 112. Moreover, the rotating frame 113 can further support various configurations not illustrated in FIG. 1. Hereinafter, in the gantry 110, the rotating frame 113 and a part, which rotationally moves with the rotating frame 113, are also referred to as a rotating part.

The X-ray high voltage device 114 has electric circuitry such as a transformer and a rectifier and has a high voltage generation device that generates a high voltage to be applied to the X-ray tube 111 and an X-ray control device that controls an output voltage corresponding to the X-rays generated by the X-ray tube 111. The high voltage generation device may be a transformer type device or an inverter type device. Note that the X-ray high voltage device 114 may be provided on the rotating frame 113, or may also be provided on a fixed frame (not illustrated).

The control device 115 has processing circuitry having a central processing unit (CPU) and the like, and a driving mechanism such as a motor and an actuator. The control device 115 receives input signals from an input interface 143 and controls the operations of the gantry 110 and the bed 130. For example, the control device 115 controls the rotation of the rotating frame 113, the tilt of the gantry 110, the operation of the bed 130, and the like. As an example, as control for tilting the gantry 110, the control device 115 rotates the rotating frame 113 around an axis parallel to the X axis direction based on information on an input inclination angle (tilt angle). Note that the control device 115 may be provided in the gantry 110 or may also be provided in the console 140.

The wedge 116 is an X-ray filter for adjusting the dose of the X-rays emitted from the X-ray tube 111. Specifically, the wedge 116 is an X-ray filter that attenuates the X-rays emitted from the X-ray tube 111 such that the X-rays emitted from the X-ray tube 111 to the subject P have a predetermined distribution. For example, the wedge 116 is a wedge filter or a bow-tie filter and is manufactured by processing aluminum and the like to have a predetermined target angle and a predetermined thickness.

The collimator 117 is a lead plate and the like for narrowing down the emission range of the X-rays having transmitted through the wedge 116 and forms a slit by a combination of a plurality of lead plates and the like. Note that the collimator 117 may also be referred to as an X-ray diaphragm. Furthermore, although FIG. 1 illustrates a case where the wedge 116 is disposed between the X-ray tube 111 and the collimator 117, the collimator 117 may be disposed between the X-ray tube 111 and the wedge 116. In such a case, the wedge 116 attenuates the X-rays, which are emitted from the X-ray tube 111 and whose emission range is limited by the collimator 117, by allowing the X-rays to pass therethrough.

The DAS 118 acquires X-ray signals detected by each detector element included in the X-ray detector 112. For example, the DAS 118 has an amplifier that performs an amplification process on electrical signals output from each detector element and an A/D converter that converts the electrical signals to digital signals and generates detection data. The DAS 118 is implemented by, for example, a processor.

The data generated by the DAS 118 is transmitted from a transmitter having a light emitting diode (LED) provided on the rotating frame 113 to a receiver having a photodiode provided on a non-rotating part (for example, a fixed frame and the like and not illustrated in FIG. 1) of the gantry 110 by optical communication and is transmitted to the console 140. The non-rotating part is, for example, a fixed frame and the like that rotatably supports the rotating frame 113. Note that the data transmission method from the rotating frame 113 to the non-rotating part of the gantry 110 is not limited to the optical communication and may adopt any non-contact type data transmission method or a contact type data transmission method.

The bed 130 is a device that places and moves the subject P to be scanned and includes a pedestal 131, a couch driving device 132, the tabletop 133, and a support frame 134. The pedestal 131 is a casing that supports the support frame 134 so as to be movable in a vertical direction. The couch driving device 132 is a driving mechanism that moves the tabletop 133, on which the subject P is placed, in a long axis direction of the tabletop 133 and includes a motor, an actuator and the like. The tabletop 133 provided on the upper surface of the support frame 134 is a plate on which the subject P is placed. Note that the couch driving device 132 may also move the support frame 134 in the long axis direction of the tabletop 133 in addition to the tabletop 133.

The console 140 has a memory 141, a display 142, the input interface 143, and processing circuitry 144. Although the console 140 is described as a separate body from the gantry 110, the gantry 110 may include the console 140 or a part of each component of the console 140.

The memory 141 is implemented by, for example, a semiconductor memory element such as a random access memory (RAM) and a flash memory, a hard disk, an optical disk, and the like. For example, the memory 141 stores a computer program for circuitry included in the X-ray CT apparatus 10 to perform its functions. Furthermore, the memory 141 stores various information obtained by imaging the subject P. Furthermore, the memory 141 stores a noise reduction processing model generated by the processing circuitry 144 to be described below. Note that the memory 141 may be implemented by a server group (cloud) connected to the X-ray CT apparatus 10 via a network.

The display 142 displays various information. For example, the display 142 displays an image containing metal artifacts to be described below. Furthermore, for example, the display 142 displays a graphical user interface (GUI) for receiving various instructions, settings, and the like from a user via the input interface 143. For example, the display 142 is a liquid crystal display or a cathode ray tube (CRT) display. The display 142 may be a desktop type display, or may be composed of a tablet terminal and the like capable of wirelessly communicating with the body of the X-ray CT apparatus 10.

Although the X-ray CT apparatus 10 is described as including the display 142 in FIG. 1, the X-ray CT apparatus 10 may include a projector instead of or in addition to the display 142. Under the control of the processing circuitry 144, the projector can perform projection onto a screen, a wall, a floor, the body surface of the subject P, and the like. As an example, the projector can also perform projection onto any plane, object, space, and the like by projection mapping.

The input interface 143 receives various input operations from a user, converts the received input operations into electrical signals, and outputs the electrical signals to the processing circuitry 144. For example, the input interface 143 is implemented by a mouse, a keyboard, a trackball, a switch, a button, a joystick, a touch pad for performing an input operation by touching an operation surface, a touch screen in which a display screen and a touch pad are integrated, non-contact input circuitry using an optical sensor, voice input circuitry, and the like. Note that the input interface 143 may be composed of a tablet terminal and the like capable of wirelessly communicating with the body of the X-ray CT apparatus 10. Furthermore, the input interface 143 may be circuitry that receives an input operation from a user by motion capture. As an example, the input interface 143 can receive a user's body movement, line of sight, and the like as an input operation by processing a signal acquired via a tracker or an image collected for a user. Furthermore, the input interface 143 is not limited to one including physical operation parts such as a mouse and a keyboard. For example, an example of the input interface 143 includes electric signal processing circuitry which receives an electric signal corresponding to an input operation from an external input device separately provided from the X-ray CT apparatus 10 and outputs the electric signal to the processing circuitry 144.

The processing circuitry 144 controls the overall operation of the X-ray CT apparatus 10 by performing a control function 144a, an imaging function 144b, an acquisition function 144c, and an output function 144f.

For example, the processing circuitry 144 reads a computer program corresponding to the control function 144a from the memory 141 and executes the read computer program, thereby controlling various functions, such as the imaging function 144b, the acquisition function 144c, and the output function 144f, based on various input operations received from a user via the input interface 143.

Furthermore, for example, the processing circuitry 144 reads a computer program corresponding to the imaging function 144b from the memory 141 and executes the read computer program, thereby imaging the subject P. For example, the imaging function 144b controls the X-ray high voltage device 114 to supply the X-ray tube 111 with a high voltage. With this, the X-ray tube 111 generates X-rays to be emitted to the subject P. Furthermore, the imaging function 144b controls the couch driving device 132 to move the subject P into an imaging port of the gantry 110. Furthermore, the imaging function 144b adjusts the position of the wedge 116 and the opening degree and position of the collimator 117, thereby controlling the distribution of the X-rays emitted to the subject P. Furthermore, the imaging function 144b controls the control device 115 to rotate the rotating part. Furthermore, while the imaging is performed by the imaging function 144b, the DAS 118 acquires X-ray signals from the respective detection elements in the X-ray detector 112 and generates detection data.

Furthermore, the imaging function 144b performs pre-processing on the detection data output from the DAS 118. For example, the imaging function 144b performs pre-processing, such as logarithmic transformation processing, offset correction processing, inter-channel sensitivity correction processing, and beam hardening correction, on the detection data output from the DAS 118. Note that the data subjected to the pre-processing is also described as raw data. Furthermore, the detection data before the pre-processing and the raw data subjected to the pre-processing are also collectively described as projection data.

Furthermore, for example, the processing circuitry 144 reads a computer program corresponding to the acquisition function 144c from the memory 141 and executes the read computer program, thereby acquiring noise data based on imaging a subject P and acquiring synthesized subject data based on first subject projection data obtained by imaging the subject P and combining with the noise data. Furthermore, for example, the processing circuitry 144 reads a computer program corresponding to the output function 144f from the memory 141 and executes the read computer program, thereby outputting a metal artifact image. Details of processing performed by the acquisition function 144c, and the output function 144f will be described below.

In the X-ray CT apparatus 10 illustrated in FIG. 1, the respective processing functions are stored in the memory 141 in the form of the computer programs executable by a computer. The processing circuitry 144 is a processor that performs a function corresponding to each computer program by reading and executing the computer program from the memory 141. In other words, the processing circuitry 144 having read the computer program has a function corresponding to the read computer program.

Note that, in FIG. 1, it has been described that the control function 144a, the imaging function 144b, the acquisition function 144c, and the output function 144f are implemented by the single processing circuitry 144, but the processing circuitry 144 may be configured by combining a plurality of independent processors, and each processor may be configured to perform each function by executing each computer program. Furthermore, each processing function of the processing circuitry 144 may be performed by being appropriately distributed or integrated into a single circuit or a plurality of processing circuits.

Furthermore, the processing circuitry 144 may also perform the functions by using a processor of an external device connected via the network. For example, the processing circuitry 144 reads and executes the computer program corresponding to each function from the memory 141 and uses, as computation resources, a server group (cloud) connected to the X-ray CT apparatus 10 via the network, thereby performing each function illustrated in FIG. 1.

Furthermore, although FIG. 1 illustrates only the single memory 141, the X-ray CT apparatus 10 may include a plurality of physically separated memories. For example, the X-ray CT apparatus 10 may separately include, as the memory 141, a memory that stores a computer program required when circuitry included in the X-ray CT apparatus 10 performs its function, and a memory that stores various information obtained by imaging the subject P.

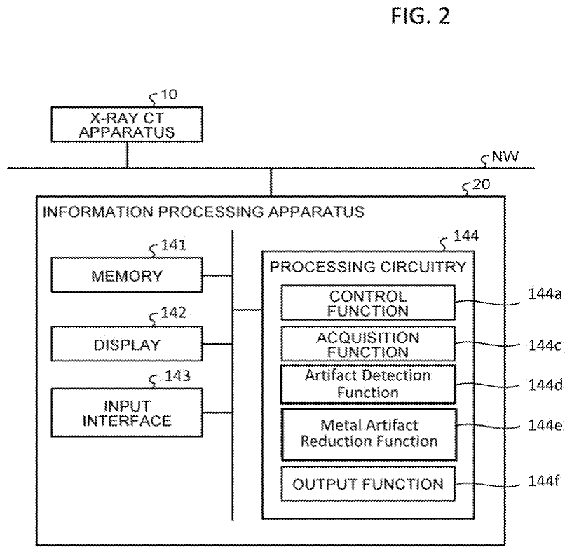

Hereinafter, this point will be described with reference to FIG. 2. FIG. 2 is a block diagram illustrating an example of a configuration of an information processing system 1 according to a second embodiment. For example, the information processing system 1 includes an X-ray CT apparatus 10 and an information processing apparatus 20 as illustrated in FIG. 2. The X-ray CT apparatus 10 and the information processing apparatus 20 are connected to each other via a network NW.

Note that the location where the X-ray CT apparatus 10 and the information processing apparatus 20 are installed is arbitrary as long as they can be connected via the network NW. For example, the X-ray CT apparatus 10 and the information processing apparatus 20 may be installed within facilities different from each other. That is, the network NW may be a local network closed within the facility or a network via the Internet. Furthermore, communication between the X-ray CT apparatus 10 and the information processing apparatus 20 may be performed via another apparatus such as an image storage apparatus, or may be directly performed without using another apparatus. An example of such an image storage apparatus includes a picture archiving and communication system (PACS) server, for example.

The X-ray CT apparatus 10 illustrated in FIG. 2 has the same configuration as that of the X-ray CT apparatus 10 illustrated in FIG. 1. However, the processing circuitry 144 of the X-ray CT apparatus 10 illustrated in FIG. 2 may or may not have such functions as the acquisition function 144c and the output function 144f. Furthermore, although FIG. 2 illustrates the X-ray CT apparatus 10 as an example of a medical image diagnostic apparatus, the information processing system 1 may include a medical image diagnostic apparatus different from the X-ray CT apparatus 10. Furthermore, the information processing system 1 may include a plurality of medical image diagnostic apparatuses.

The information processing apparatus 20 performs various processes based on data acquired by the X-ray CT apparatus 10. For example, as illustrated in FIG. 2, the information processing apparatus 20 includes a memory 141, a display 142, an input interface 143, and processing circuitry 144. The display 142 can be configured similarly to the aforementioned display 142 in the apparatus 10. The information processing apparatus 20 may include a projector instead of or in addition to the display 142.

The input interface 143 can be configured similarly to the aforementioned input interface 143 of the X-ray CT apparatus 10. For example, the input interface 143 receives various input operations from a user, converts the received input operations into electrical signals, and outputs the electrical signals to the processing circuitry 144.

The processing circuitry 144 controls the overall operation of the information processing apparatus 20 by performing a control function 144a, an acquisition function 144c, and an output function 144f. For example, the control function 144a controls various functions such as the acquisition function 144c and the output function 144f based on the various input operations received from the user via the input interface 143. The acquisition function 144c is a function corresponding to the acquisition function 144c of the X-ray CT apparatus 10. The output function 144f is a function corresponding to the output function 144f of the X-ray CT apparatus 10.

In the information processing apparatus 20 illustrated in FIG. 2, respective processing functions are stored in the memory 141 in the form of computer programs that can be executed by a computer. The processing circuitry 144 is a processor that reads and executes the computer programs from the memory 141, thereby performing functions corresponding to the computer programs. In other words, the processing circuitry 144 having read the computer programs has the functions corresponding to the read computer programs. Furthermore, each processing function of the processing circuitry 144 may be performed by being appropriately distributed or integrated into a single processing circuit or a plurality of processing circuits. Furthermore, the processing circuitry 144 may also perform the functions by using a processor of an external device connected via the network NW. For example, the processing circuitry 144 reads and executes the computer programs corresponding to the functions from the memory 141 and uses, as computation resources, a server group (cloud) connected to the information processing apparatus 20 via the network NW, thereby performing the functions illustrated in FIG. 2.

Furthermore, in FIG. 1, it has been described that the single memory 141 stores the computer programs corresponding to the respective processing functions of the processing circuitry 144. Furthermore, in FIG. 2, it has been described that the single memory 144 stores the computer programs corresponding to the respective processing functions of the processing circuitry 144. However, the embodiment is not limited thereto. For example, a plurality of memories 141 may be arranged in a distributed manner, and the processing circuitry 144 may be configured to read corresponding computer programs from the individual memories 141. Furthermore, instead of storing the computer programs in the memory 141, the computer programs may be directly incorporated in the circuit of the processor. In such a case, the processor reads and executes the computer programs incorporated in the circuit to perform functions thereof.

Each component of each apparatus according to the aforementioned embodiment is functionally conceptual and does not necessarily need to be physically configured as illustrated in the drawings. That is, the specific form of distribution and integration of each apparatus is not limited to that illustrated in the drawing and all or some thereof can be functionally or physically distributed and integrated in arbitrary units according to various loads, usage conditions, and the like. Moreover, all or some of the processing functions performed by each apparatus may be performed by the CPU and the computer programs that are analyzed and executed by the CPU, or may be performed as a wired logic-based hardware.

Furthermore, the information processing method described in the aforementioned embodiment can be implemented by executing an information processing program prepared in advance on a computer such as a personal computer and a workstation. The information processing program can be distributed via a network such as the Internet. Furthermore, the information processing program can be executed by being recorded on a non-transitory computer readable recording medium such as a hard disk, a flexible disk (FD), a CD-ROM, an MO, and a DVD, and being read from the recording medium by the computer.

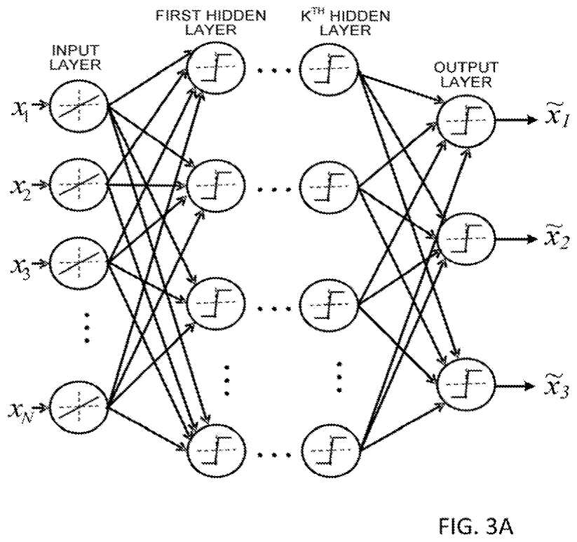

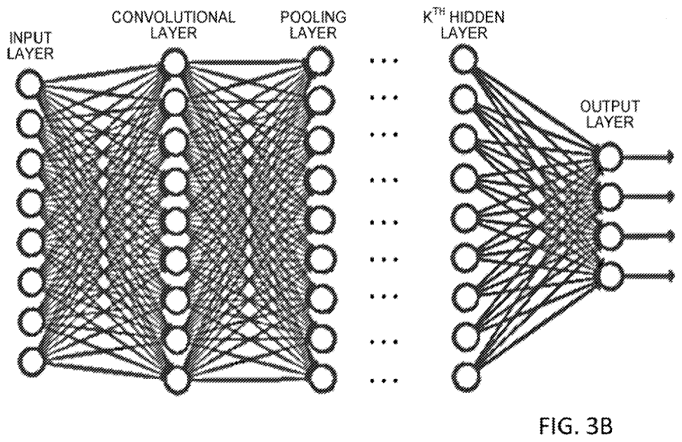

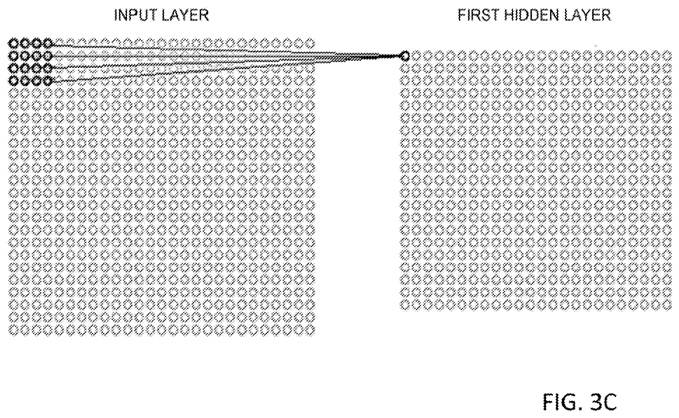

FIG. 3A to FIG. 3C illustrate a training process according to an exemplary embodiment described below. More specifically, FIG. 3A illustrates a general artificial neural network (ANN) having n inputs, a K^{th} hidden layer, and three outputs. Each layer of the ANN is made up of nodes (also called neurons), and each node performs a weighted sum of the inputs to produce an output and compares the result of the weighted sum with a threshold. ANNs make up a class of functions for which members of the class are acquired by varying thresholds, connection weights, or specifics of an architecture such as the number of nodes and/or their connectivity. The nodes in the ANN may be referred to as neurons (or neuronal nodes), and the neurons can have inter-connections between different layers of the ANN system. For example, the ANN has more than three layers of neurons and has as many output neurons x to N as input neurons, wherein N is the number of pixels in the reconstructed image. Synapses (that is, connections between neurons) store values called “weights” (also interchangeably referred to as “coefficients” or “weighting coefficients”) that manipulate data in calculations. The outputs of the ANN depend on three types of parameters: (i) An interconnection pattern between different layers of neurons, (ii) A learning process for updating weights of the interconnections, and (iii) An activation function that converts a neuron's weight input to its output activation.

Mathematically, a neuron's network function m(x) is defined as a composition nᵢ(x) of other functions, which can further be defined as a composition of other functions. This can be conveniently represented as a network structure, with arrows depicting dependencies between variables, as illustrated in FIG. 3A. For example, the ANN can use a nonlinear weighted sum, wherein m (x)=K(Σᵢwᵢnᵢ(x)), where K (commonly referred to as an “activation function”) is a predetermined coefficient such as a sigmoidal function, a hyperbolic tangent function, and a rectified linear unit (ReLU).

In FIG. 3A (and similarly in FIG. 3B), the neurons (that is, nodes) are depicted by circles around a threshold function. In the non-limiting example illustrated in FIG. 3A, the inputs are depicted by circles around a linear function and the arrows indicate directed connections between neurons. In a specific embodiment, the ANN is a feedforward network as exemplified in FIG. 3A and FIG. 3B (for example, it can be represented as a directed acyclic graph).

The ANN operates to achieve a specific task, such as identification of a CT image containing a metal artifact, by searching within the class of a function F to learn, using a set of observation results, to find an element m* (m*∈F) which solves the specific task in some optimal criteria (for example, a stopping criteria). For example, in a specific embodiment, this can be achieved by defining a cost function C:F→R, such as for an optimal solution expressed by the following Equation (1) (that is, no solution having a cost less than the cost of the optimal solution).

C(m*)≤C(m)∀m∈F (1)

In Equation (1), m* is the optimal solution. The cost function C is a measure of how far away a particular solution is from an optimal solution to a problem to be solved (for example, an error). Learning algorithms iteratively search through the solution space to fine a function with the smallest possible cost. In a specific embodiment, the cost is minimized over a sample of the data (that is, the training data).

FIG. 3B illustrates a non-limiting example in which the ANN is a DCNN. The DCNN is a type of ANN having beneficial properties for image processing. The DCNN uses a feedforward ANN in which a connectivity pattern between neurons can represent convolutions in image processing. For example, the DCNN can be used for image processing optimization by using multiple layers of small neuron collections that process portions of an input image, called receptive fields. The outputs of these collections can then be tiled so that they overlap, to achieve a better representation of the original image. This processing pattern can be repeated over multiple layers having alternating convolution and pooling layers. Note that FIG. 3B illustrates an example of a fully connected (full connect) network that defines a node of a succeeding layer by using all the nodes of a preceding layer. This example only illustrates an example of a deep neural network (DNN). It is common for the DCNN to form a loosely connected (partial connect) network that defines a node of a succeeding layer by using some of the nodes of a preceding layer.

FIG. 3C illustrates an example of a 5×5 kernel being applied to map values from an input layer representing a two-dimensional image to a first hidden layer which is a convolution layer. The kernel maps respective 5×5 pixel regions to corresponding neurons of the first hidden layer. Following after the convolution layer, the DCNN can include local and/or global pooling layers that combine the outputs of neuron clusters in the convolution layers. Moreover, in a specific embodiment, the DCNN can also include various combinations of convolutional and fully connected layers, with pointwise nonlinearity applied at the end of or after each layer.

The DCNN has several advantages for image processing. To reduce the number of free parameters and improve generation, a convolution operation on small regions of input is introduced. One significant advantage of the specific embodiment of the DCNN is the use of shared weights in the convolution layer, that is, filters (weight banks) used as coefficients for each pixel in the layer are the same. Such significant advantages reduce a memory footprint and improve performance. Compared to other image processing methods, the DCNN advantageously uses relatively little pre-processing. This means that the DCNN is responsible for learning manually designed filters in traditional algorithms. The lack of dependence on prior knowledge and human effort in designing features is a major advantage for the DCNN.

In the supervised learning, a set of training data is acquired, and the network is iteratively updated to reduce errors, such that output of the partially trained network improves to match a desired/target output using a cost function The cost function can use a mean-squared error to optimize an average squared error. In the case of multilayer perceptrons (MLP) neural network, a backpropagation algorithm can be used for training the network by minimizing the mean-squared-error-based cost function using a gradient descent method. In general, DL networks can be trained using any of numerous algorithms for training neural network models (for example, applying optimization theory or statistical estimation).

For example, the optimization method used in training artificial neural networks can use some form of gradient descent, using backpropagation to compute actual gradients. This is done by taking the derivative of the cost function with respect to network parameters and then changing those parameters in a gradient-related direction. The backpropagation algorithm may be a steepest descent method (for example, with variable learning rate, with variable learning rate and momentum, and resilient backpropagation), a quasi-Newton method (for example, Broyden-Fletcher-Goldfarb-Shanno, one step secant, and Levenberg-Marquardt), or a conjugate gradient method (for example, Fletcher-Reeves update, Polak-Ribiére update, Powell-Beale restart, and scaled conjugate gradient). Moreover, evolutionary methods, such as gene expression programming, simulated annealing, expectation-maximization, non-parametric methods, and particle swarm optimization, can also be used for training the DCNN.

When the cost function (for example, the error) has a local minimum different from the global minimum, a robust stochastic optimization process is beneficial to find the global minimum of the cost function. An example of an optimization method for finding a local minimum can be a Nelder-Mead simplex method, a gradient descent method, a Newton's method, a conjugate gradient method, a shooting method, and one of other known local optimization methods. There are also many known methods for finding global minima, including generic algorithms, simulated annealing, exhaustive searches, interval methods, and other related deterministic, stochastic, heuristic, and metaheuristic method. Any of these methods can be used to optimize the weights/coefficients of the DCNN. Moreover, neural networks can also be optimized using a backpropagation method.

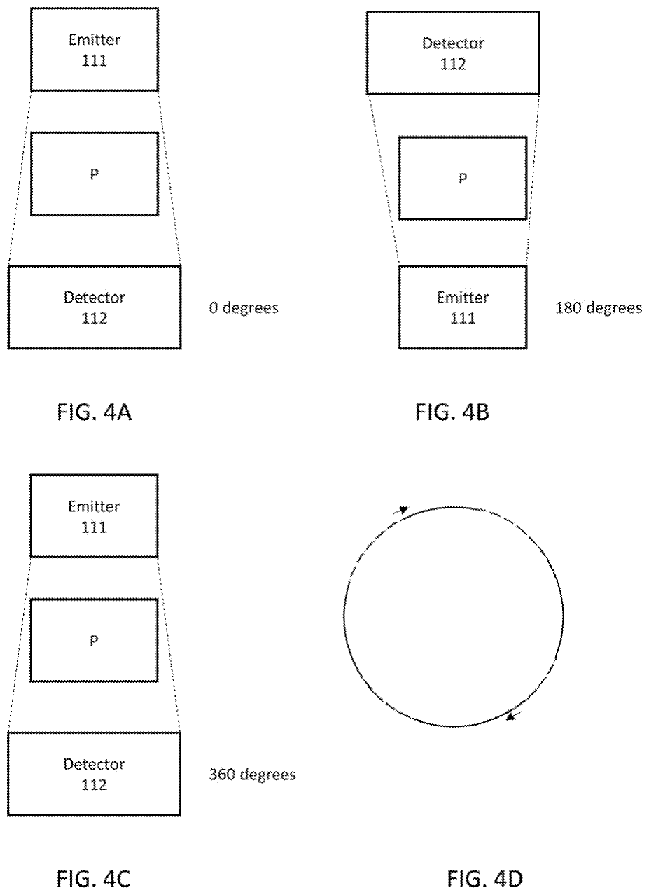

Using a CT apparatus 10 (in FIGS. 1 and 2), an emitter 111 can be used to obtain first lower-radiation dose three-dimensional image data during a first scan of a patient as would be performed during a scout scan or a preliminary scan prior to a main scan (or a diagnostic scan). The process is generally shown starting in FIG. 4A where a scanner starts at a first angle and obtains image data at least until a second angle as shown in FIG. 4B. By obtaining data through the second angle (e.g., 180 degrees), the system can perform a half-reconstruction so that three-dimensional information about the patient can be generated. As shown in FIG. 4C, data can be obtained for more than 180 degrees, and by obtaining for more than 180 degrees, higher definition image reconstruction can be performed. Although illustrated as stopping at 180 degrees or 360 degrees, other stopping angles can be used (e.g., when an imaging apparatus is a limited extent apparatus that rotates less than 180 degrees such as in a limited extent tomosynthesis device).

In one embodiment, image data is reconstructed image data. In an alternate embodiment, image data is projection-domain data (sinogram data).

In addition, it is possible to use sparse mode acquisition during the three-dimensional scout scan to further reduce radiation administered to a patient. In such a case, the system uses a sparse mode reconstruction technique that performs extrapolation between views where needed.

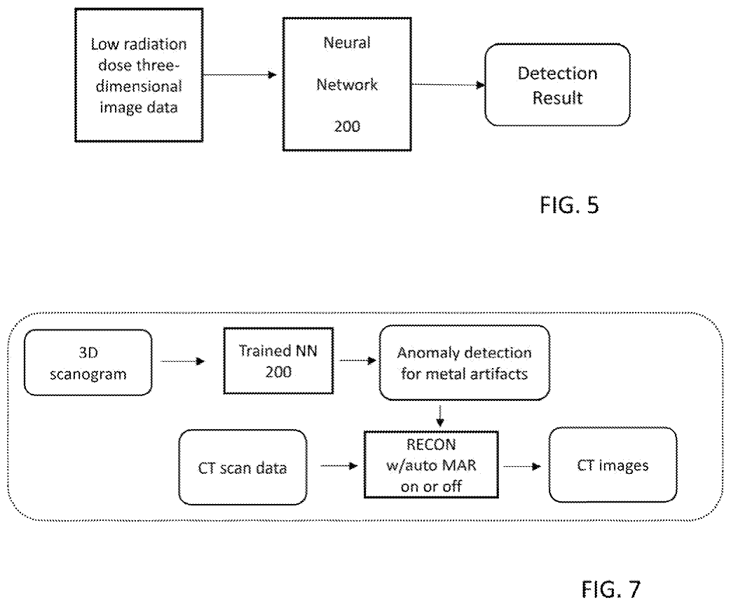

Having obtained the first lower-radiation dose image data during the first scan, the method and system described herein can be used to detect a presence of one or more materials and/or the image degradation caused by the presence of the at least one material. For example, as shown in FIG. 5, a trained network 200 can be used to detect a presence of an artifact in the image data by detecting an image artifact present in the first lower-radiation dose image data.

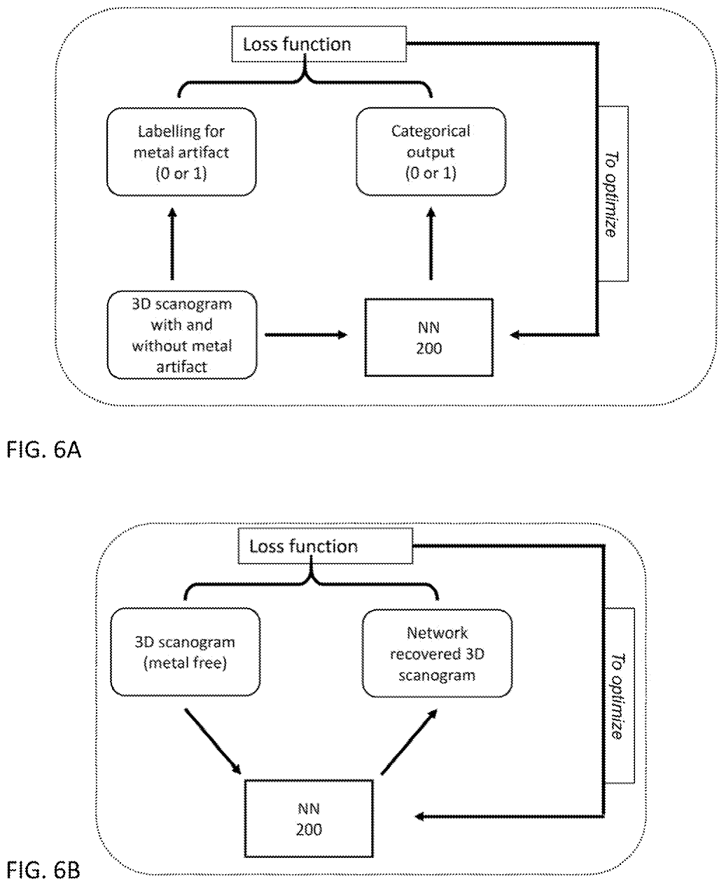

According to one aspect described herein, a neural network 200 as shown in FIG. 6A can be trained to perform a binary classification task, where the untrained network is trained with “0” and “1” labelled targets so that it could learn critical features to differentiate between image data (in either or both of the image domain and the projection domain) with and without metal artifacts. In another aspect, aspect, the neural network 200 can be trained as a classification network to output a detection result indicating the presence of a number of different materials (e.g., outputting 0 for no artifact materials, 1 for the presence of a metal artifact, and 2 for the presence of a streak artifact, and 3 for the presence of another artifact). During the training of the network 200, in FIG. 6A an untrained network is provided the lower-radiation dose image data obtained from at least one angle (but potentially obtained from a sufficient number of angles to produce any of the reconstructions described herein) and trained to minimize a loss function which matches a labelled result (a result known a priori) with a calculated result. For example, 100 sets of image data without a metal artifact and 100 sets of image data with a metal artifact are provided to the untrained network and the untrained network is given the correct classification of each as label data and trained to predict whether image data other than the training data includes the presence of the same kind of artifact. The amount of positive and negative image data may need to be increased to achieve proper results.

Alternatively, a neural network other than a classifier neural network can be used. As shown in FIG. 6B, by applying the lower-radiation dose image data obtained from at least one angle (but potentially obtained from a sufficient number of angles to produce any of the reconstructions described herein), an autoencoder can be used to autoencode image data without artifacts to produce a trained autoencoder. When new artifact-free image data is applied to the trained autoencoder after training, then the autoencoder will generate a regenerated version of the input image data that is similar to the input image data (that is either image-domain based data or projection domain data). However, when the trained autoencoder generates a regenerated version from input image data that includes artifacts, then the regenerated version will be sufficiently different from the input image data that a comparison of the two versions indicates the presence of an artifact. For example, when a difference between the original image data and the regenerated image data is larger than a threshold, then the original image data will be considered to include an artifact. such a threshold can be determined empirically by calculating a threshold value for which manually labelled data including artifacts are applied to the trained network and compared to their regenerated versions having been applied to a trained network.

FIG. 7 illustrates part of an image processing method whereby the presence of a detected artifact causes automatic artifact reduction processing to be performed (and by-passed if no artifact is detected). Thus, the system and method can avoid processing data from a full scan that is detected to have an artifact until after artifact reduction processing can be performed on the full data.

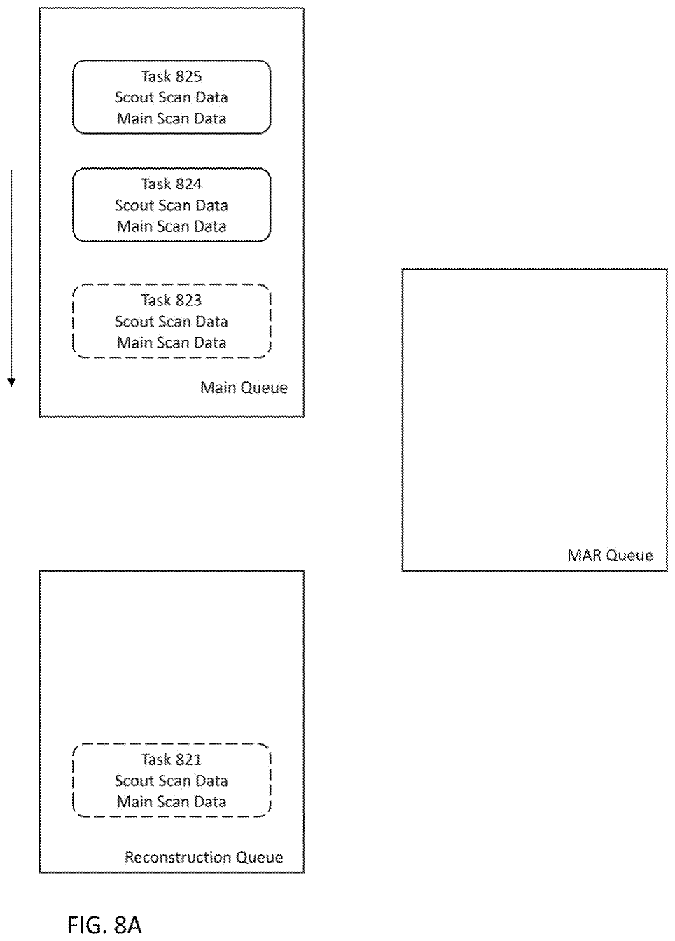

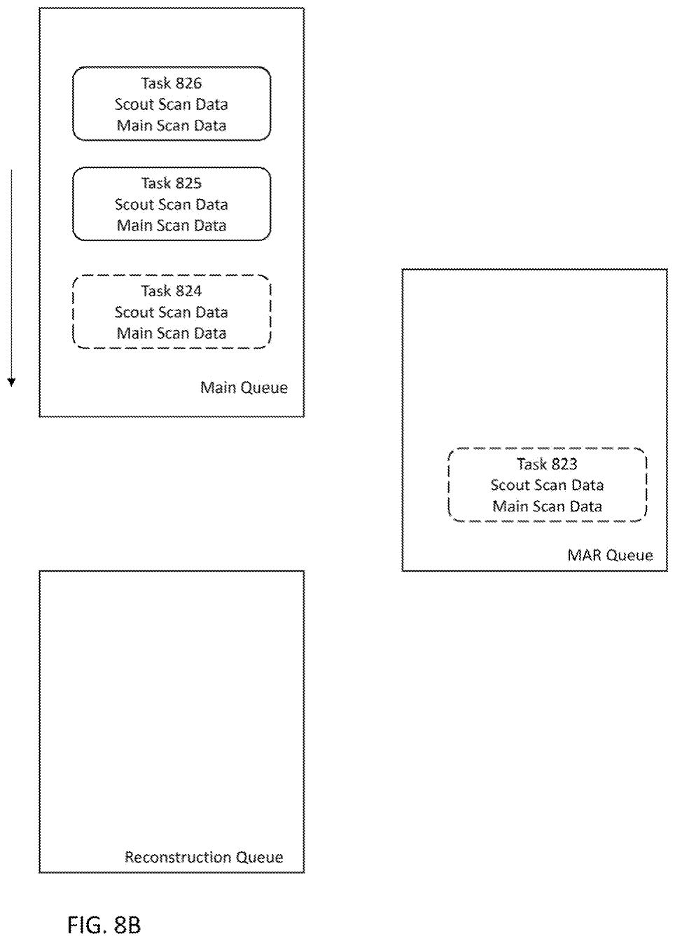

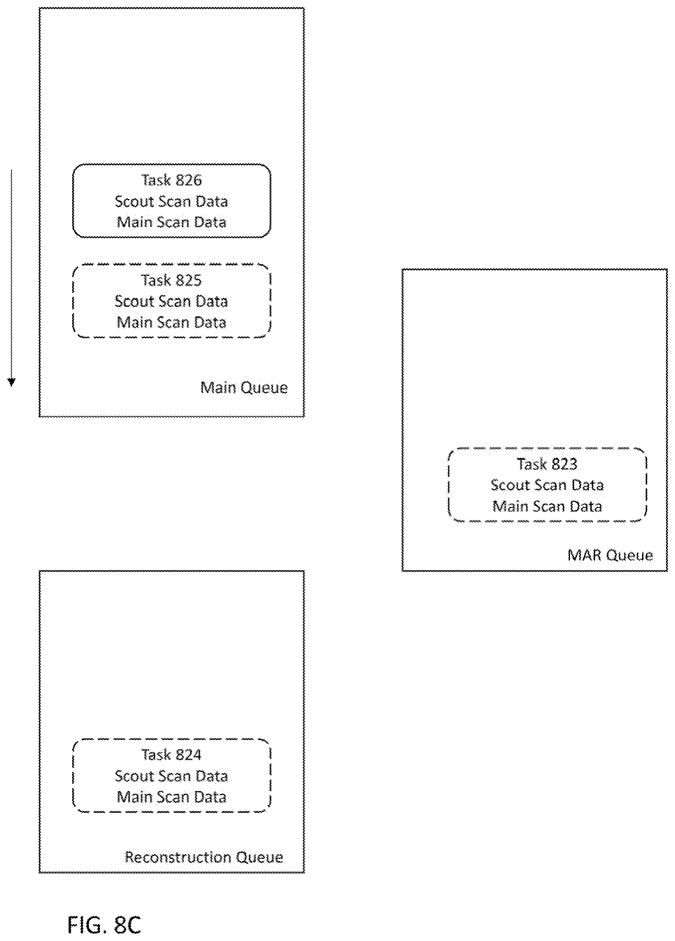

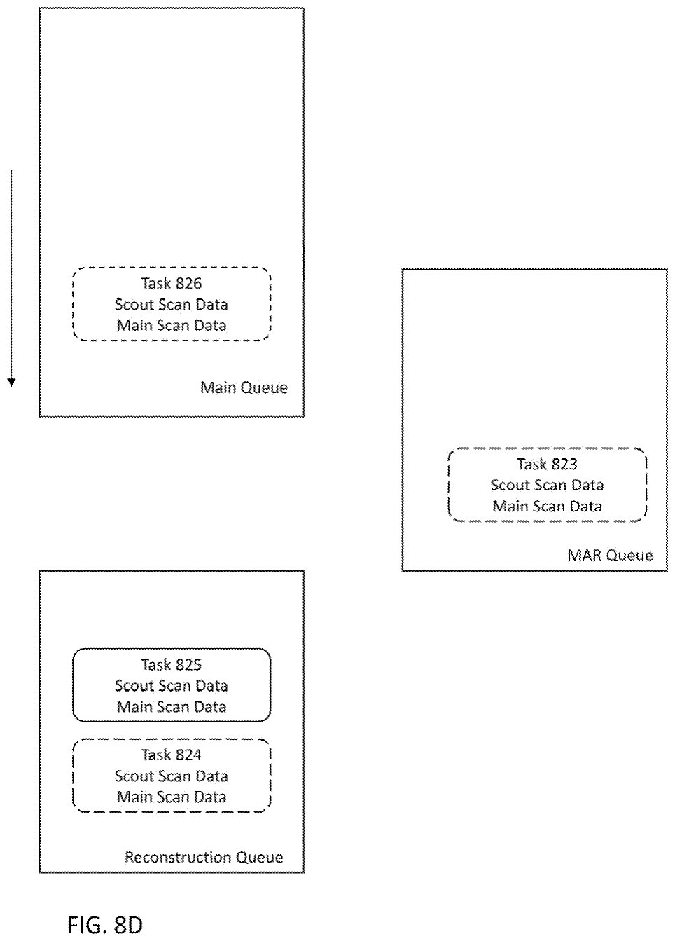

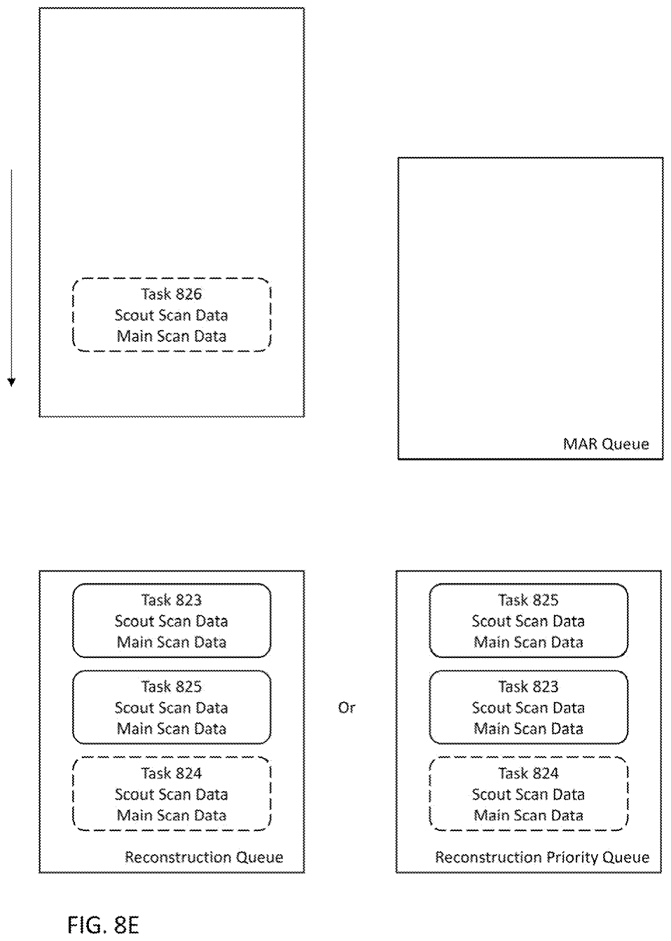

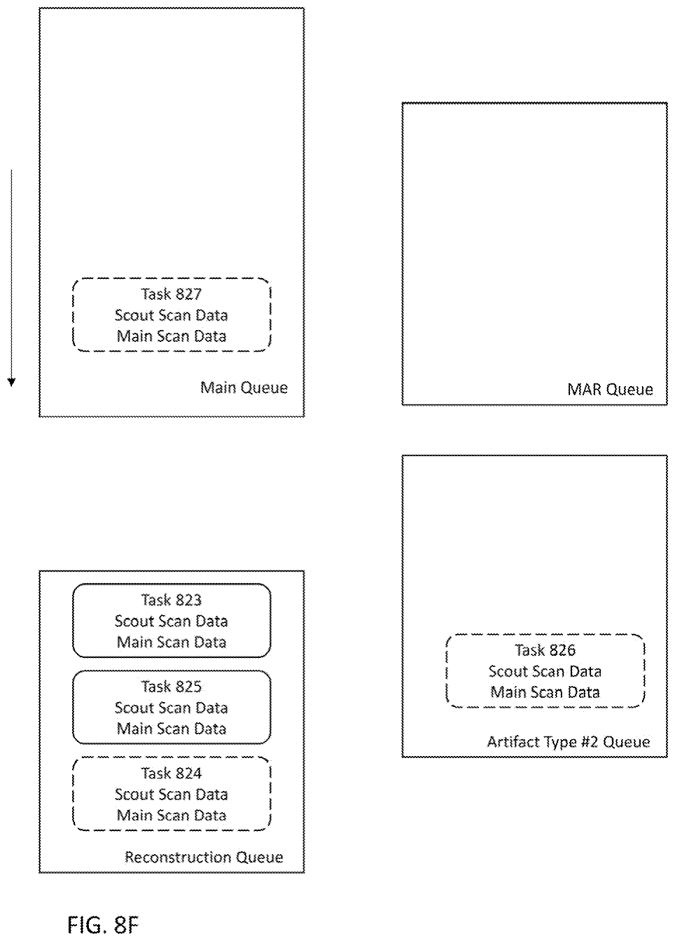

FIGS. 8A-8F shows a set of queues that can be used by an automatic image processing system to process the scout scans and main scans of FIG. 7. In FIGS. 8A-8F, the dashed rounded rectangles indicate tasks that are currently being processed in the queue, and the solid rounded rectangles indicate tasks that are waiting to be executed. The tasks each are indicated as including scout scan data and main scan data. Such data may either be explicitly included in the task (e.g., as part of a file being processed by the processor implementing the main queue) or may be logically included in the task by including a file locator that enables the main queue to know from where the data should be retrieved.

As shown in FIG. 8A, a main queue originally has three tasks in it (e.g., Tasks 823, 824, and 825), a Metal Artifact Reduction (MAR) Queue us initially empty, and a reconstruction queue initially contains a single task 821. Tasks are automatically assigned to the main queue when each main image acquisition process is complete. As task 823 is processed in the main queue, the system determines whether the scout scan shows that at least one artifact is present in the scout scan data of task 823. If so, the task is added to the MAR queue and then next task in the queue (e.g., task 824) is processed as shown in FIG. 8B. As shown in FIG. 8C, if the scout scan of Task 824 is found not to include an artifact, then the system can automatically add the corresponding task directly to the reconstruction queue without waiting for the MAR queue to finish with Task 823. Depending on how long the MAR processing takes compared with the reconstruction processing, multiple tasks may be processed from the main queue and added to the reconstruction queue before Task 823 has finished in the MAR queue. FIG. 8D shows two tasks that were initially behind task 823 now in the reconstruction queue before it.

As shown in FIG. 8E, when task 823 has completed its MAR processing, it is automatically added to the reconstruction queue. In an embodiment where the reconstruction queue is a first in first out (FIFO) queue, Task 823 is simply added behind those tasks that were already added before it. Alternatively, the reconstruction queue can be implemented as a priority queue such that specified condition (e.g., the task number) controls a location to which the task is added. Other possible conditions can be used to change an order in a priority queue. For example, if a set of reconstructions are flagged as important, a “priority flag” can be added to a task to make it next in line to be processed.

As shown in FIG. 8F, the processing described herein is not limited to a single type of artifact, and scout scans with artifacts can be processed within the same queue or different queues. The reconstruction processing and each of the artifact reduction processings can be performed on the same processor or on different processors. For example, artifact processing may be offloaded to a special purpose computer (using different hardware and/or software) that is programmed to perform artifact processing not performed by the computer performing reconstruction.

The term “processor” used in the above description, for example, means a circuit such as a CPU, a graphics processing unit (GPU), an application specific integrated circuit (ASIC), and a programmable logic device (for example, a simple programmable logic device (SPLD), a complex programmable logic device (CPLD), and a field programmable gate array (FPGA)). When the processor is, for example, the CPU, the processor performs functions by reading and executing computer programs stored in a storage circuit. On the other hand, when the processor is, for example, the ASIC, the functions are directly incorporated in the circuit of the processor as a logic circuit instead of storing the computer programs in the storage circuit. Note that each processor of the embodiment is not limited to a case where each processor is configured as a single circuit, and one processor may be configured by combining a plurality of independent circuits to perform functions thereof. Moreover, a plurality of components in each drawing may be integrated into one processor to perform functions thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method of processing an x-ray image, comprising:
obtaining first lower-radiation dose three-dimensional image data during a first scan of a patient; and
detecting, using a trained neural network, a presence of an artifact in the first lower-radiation dose three-dimensional image data.

2. The method according to claim 1, wherein the artifact is a metal artifact.

3. The method according to claim 1, wherein the trained neural network is a trained binary classification neural network trained to detect the presence of the artifact.

4. The method according to claim 1, wherein the trained neural network is a trained classification neural network trained to detect a presence of plural artifacts of different materials.

5. The method of according to claim 1, further comprising:
obtaining second higher-radiation dose three-dimensional image data during a second scan of the patient; and
applying artifact correction to the second higher-radiation dose three-dimensional image data if the trained neural network detects the presence of the artifact.

6. The method according to claim 1, wherein the first lower-radiation dose three-dimensional image data obtained during the first scan comprises three-dimensional image data for performing a half-scan reconstruction.

7. The method according to claim 1, wherein the first lower-radiation dose three-dimensional image data obtained during the first scan comprises three-dimensional image data for performing a full-scan reconstruction.

8. The method according to claim 1, wherein the first lower-radiation dose three-dimensional image data obtained during the first scan comprises three-dimensional image data for performing a sparse reconstruction.

9. An image processing apparatus, comprising:
processing circuitry configured to:
obtain first lower-radiation dose three-dimensional image data during a first scan of a patient; and
detect, using a trained neural network, a presence of an artifact in the first lower-radiation dose three-dimensional image data.

10. The image processing apparatus according to claim 9, wherein the artifact is a metal artifact.

11. The image processing apparatus according to claim 9, wherein the trained neural network is a trained binary classification neural network trained to detect the presence of the artifact.

12. The image processing apparatus according to claim 9, wherein the trained neural network is a trained classification neural network trained to detect a presence of plural artifacts of different materials.

13. The image processing apparatus according to claim 9, wherein the processing circuitry is further configured to:
obtain second higher-radiation dose three-dimensional image data during a second scan of the patient; and
apply artifact correction to the second higher-radiation dose three-dimensional image data if the trained neural network detects the presence of the artifact.

14. The image processing apparatus according to claim 9, wherein the first lower-radiation dose three-dimensional image data obtained during the first scan comprises three-dimensional image data for performing a half-scan reconstruction.

15. The image processing apparatus according to claim 9, wherein the first lower-radiation dose three-dimensional image data obtained during the first scan comprises three-dimensional image data for performing a full-scan reconstruction.

16. The image processing apparatus according to claim 9, wherein the first lower-radiation dose three-dimensional image data obtained during the first scan comprises three-dimensional image data for performing a sparse reconstruction.

17. The image processing apparatus according to claim 9, further comprising:
an x-ray transmitter and an x-ray detector for acquiring the first lower-radiation dose three-dimensional image data during the first scan of a patient.

18. A computer storage device, comprising:
a non-transitory computer readable medium for storing computer instructions, wherein the computer instructions cause a computer processor, when reading out the computer instruction from a computer memory and executing the computer instructions, to perform a method comprising:
obtaining first lower-radiation dose three-dimensional image data during a first scan of a patient; and
detecting, using a trained neural network, a presence of an artifact in the first lower-radiation dose three-dimensional image data.
